(19) United States
(12) Patent Application Publication
(10) Pub. No.: US 2006/0144407 A1
(43) Pub. Date: Jul. 6, 2006

(54) Magnetic navigation manipulation apparatus

(76) Inventors: Anthony Aliberto, Laguna Hills, CA (US); Paul Burmeister, Maple Grove, MN (US); Richard DiMonda, Marietta, GA (US)

Correspondence Address: HARNESS, DICKEY, & PIERCE, P.L.C, 7700 BONHOMME, STE 400, ST. LOUIS, MO, 63105, US

(21) Appl. No.: 11/185,438

(22) Filed: Jul. 20, 2005

Related U.S. Application Data
(60) Provisional application No. 60/589,468, filed on Jul. 20, 2004.

(51) Int. Cl. A61B 19/00

(52) U.S. Cl. 128/899; 600/431

(57) ABSTRACT

The inventive apparatus may be inserted into a guiding catheter or other thru-lumen catheters and tubing to assist in navigating and placing the distal end of the guiding catheter or other thru-lumen catheters and tubing at a target location within a patient, by utilizing an externally applied magnetic field to align a plurality of magnetically responsive elements on the apparatus towards the target location. The guiding catheter or other thru-lumen catheters and tubing is then advanced and guided by the apparatus, which facilitates access of the guiding catheter or other thru-lumen catheters and tubing to the target vessel to save time during the catheter placement procedure. The apparatus in combination with magnetic navigation also provides support to hold the guiding catheter or other thru-lumen catheters and tubing in place to resist back out of the catheter.

### CROSS-REFERENCE TO RELATED APPLICATIONS

This application claims the benefit of U.S. Provisional Patent Application Ser. No. 60/589,468, filed Jul. 20, 2004. The disclosure of the above-referenced application is incorporated herein by reference.

### FIELD OF THE INVENTION

This invention relates to guide catheters, or other thru-lumen catheters and tubing and more particularly to devices for use in guide catheters or other thru-lumen catheters and tubing that may be magnetically steered within the body.

### BACKGROUND OF THE INVENTION

Guiding catheter devices, for example, have been used as a conduit for delivery of therapeutic tools into specific regions of the body, and may be manually guided by a physician to gain access to specific points in the vasculature system of a patient. For example, a guide catheter used in Angioplasty may be inserted in the patient's arterial system through a puncture of the femoral artery, and a torque applied to the proximal end of the guide catheter to rotate the distal end while pushing the guide catheter. This action is repeated until, by trial and error, the guide catheter distal tip enters the desired vessel branch. Such trial and error methods can cause additional vessel wall contact in trying to reach the desired target location, potentially injuring the vessel wall. Some guide catheters have a pre-shaped end structure that aids in navigating the distal end of the catheter, and allows the mechanical pushing forces to be directed to the distal end of the catheter in a selected direction. However, physicians often experience back out of the guide catheter from the intra-arterial location, where the tip of the guide catheter moves away from its target location after being positioned there by the physician. Advancing guide wires or other medical devices through the guide catheter can also contribute to the back out of the guide catheter due to opposing forces, for example. Thus, there is a need for a device and method for positioning a guide catheter at a desired location in the vasculature of a patient, and for holding and anchoring the guide catheter in the desired location.

### SUMMARY OF THE INVENTION

The present invention relates to an apparatus and method for magnetically navigating the distal end of a guiding catheter or other thru-lumen catheters and tubing to a desired location within the vasculature of a patient, and for holding the guiding catheter or other thru-lumen catheters and tubing to resist back out of the catheter from the desired location. In one embodiment, the apparatus comprises a flexible tube having a proximal end and a distal end, a lumen therebetween, and a plurality of magnetically responsive elements disposed on the distal end of the apparatus, whereby an externally applied magnetic field can be used to preferentially align the magnetically responsive elements to guide the distal end through various parts of the vasculature. In one preferred embodiment, a set of adjacently positioned magnetically responsive elements are located within a minimum distance from the distal end of the flexible tube, and one or more magnetically responsive elements are proximally spaced from the set of magnetically responsive elements by one or more predetermined distances. The predetermined distances provide regions of flexibility for enabling the distal end of the apparatus to deflect at least a minimum angle from the longitudinal axis of the tube when subjected to an external magnetic field. In use, the distal end of the apparatus may be inserted into the guide catheter or other thru-lumen catheters and tubing, and advanced beyond the distal tip of the guide catheter or other thru-lumen catheters and tubing towards the ostium of an artery, for example, where the distal end of the apparatus may be magnetically navigated inside the ostium of the target vessel. The apparatus remains in place, and a guide catheter, for example, may be guided by the apparatus into the ostium of the target vessel. Guide wires and other medical devices are then able to travel through the inside of the apparatus and out the end to the desired vessel or target area. The apparatus in combination with magnetic navigation facilitates access to the target vessel to save time during the guide catheter placement procedure, as an example, and assists in holding the guide catheter in place to resist back out of the catheter.

According to one aspect of the invention, there is provided an apparatus and method for controlling navigation and placement of the distal end of a guide catheter or other thru-lumen catheters and tubing at the target location within the vasculature of a patient, which method utilizes an externally applied magnetic field to align the plurality of magnetically responsive elements on the distal end of the apparatus in a desired path to access the target location. The magnetically navigable apparatus therefore provides guidance of the distal end of the guide catheter or other thru-lumen catheters and tubing to the desired target location.

Embodiments of the invention control the position of a guide catheter or other thru-lumen catheters and tubing to resist back out of the catheter from its position within the vasculature, by utilizing an applied magnetic field to align the magnetic members on the distal end of the apparatus so as to hold the apparatus and catheter in place.

### BRIEF DESCRIPTION OF THE DRAWINGS

FIG. 1 is a side elevation view of the magnetically navigable maneuvering apparatus in accordance with the principles of the present invention;

FIG. 2 is an illustration of the magnetically navigable maneuvering apparatus inserted through a guide catheter, in accordance with the principles of the present invention.

### DETAILED DESCRIPTION OF THE INVENTION

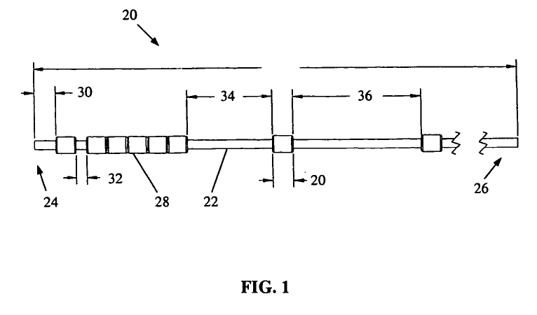

An apparatus for magnetically guiding the distal end of a guide catheter or other thru-lumen catheters and tubing to a target location within a subject's body in accordance with the principles of the present invention is indicated generally as 20 in FIG. 1. The apparatus 20 comprises a flexible element 22 having a proximal end 24 and a distal end 26. The flexible element 22 is preferably a tube made of Pebax, but may alternatively be made from Nylon, Polyolefin, PET, Polyurethane, Silicone or other thermoplastic materials providing suitable flexibility. The apparatus 20 further comprises a plurality of magnetically responsive elements 28 disposed around the tube 22 near the distal end 26, wherein the magnetically responsive elements align with an externally applied magnetic field to align the distal end of the apparatus in a desired direction.

In a preferred embodiment, a first magnetically responsive element 28 is located at a minimum distance 30 from the distal end 26 of the flexible tube 22, and is preferably secured to the tube 22 with an adhesive or other suitable bonding method. The minimum distance 30 of the preferred embodiment is preferably about 1 millimeter, but may alternately be any distance that achieves the desired flexibility and magnetic navigation characteristics. It should be noted that the apparatus may alternatively be constructed without the first magnetically responsive element. In the preferred embodiment, a predetermined number of magnetically responsive elements 28 are positioned adjacent to each other and are located at a minimum distance 32 from the first magnetically responsive element. This minimum distance 32 of the preferred embodiment is preferably about 1 millimeter from the first magnetically responsive element, but may alternatively be about 3 to 5 millimeters from the distal end of the tube 22 in an alternate construction omitting the first magnetically responsive element. The predetermined number of adjacent magnetically responsive elements is preferably in the range of between 1 to 6, depending in part upon the size of the magnetically responsive elements. The magnetically responsive elements are sized such that the magnetically responsive elements on the distal end of the apparatus align the distal end with an externally applied magnetic field of as low as 0.1 Tesla, and more preferably as low as 0.06 Tesla. In the preferred embodiment the size is preferably not more than about 20 millimeter in outside diameter and preferably no longer than 10 millimeters in length. The outside diameter is sized to allow the apparatus to be inserted through the lumen of most commercially available guiding catheters or other thru-lumen catheters and tubing having a typical inside diameter ranging from 0.25 millimeters to 20 millimeters. The magnetically responsive elements 28 in this guiding catheter example, may be of a length in the range of 1.5 to 3.0 millimeters, which may accordingly vary the predetermined number of magnetically responsive elements positioned adjacent to each other. Although designated with a single reference numeral, the magnetically responsive elements do not all have to be of the same size and shape.

The 20 apparatus further comprises one or more magnetically elements 28 proximally spaced from the predetermined number of adjacently positioned magnetically responsive elements, wherein the one or more magnetically responsive elements 28 are spaced at one or more predetermined distances. The predetermined distances 34 and 36 provide regions of flexibility in the apparatus 20 for enabling the distal end of the apparatus to deflect a minimum angle from the longitudinal axis of the tube 22 when subjected to an external magnetic field. In the preferred embodiment, in this example, the apparatus comprises a first magnetically responsive element 28 spaced proximally from the predetermined number of adjacent magnetically responsive elements at a distance 34 in the range of 8 to 10 millimeters, and further comprises a second magnetically responsive element 38 spaced proximally from the first magnetically responsive element at a distance 36 in the range of 12 to 14 millimeters in length. The distance to the second magnetically responsive element 28 may also be expressed as a distance of 20 to 24 millimeters from the predetermined number of adjacent magnetically responsive elements.

When the plurality of magnetically responsive elements 28 at the distal end of the apparatus 20 are subjected to an externally applied magnetic field, the magnetically responsive elements substantially align the distal tip with the direction of an externally applied magnetic field. The magnetically responsive elements 28 can be made of a permanent magnetic material or a permeable magnetic material. The magnetically responsive elements 28 are made of such material and are of such dimensions that under the influence of an applied magnetic field, the distal end portion of the apparatus substantially aligns with the local applied magnetic field direction. In the preferred embodiment the magnetically responsive elements 28 or allow the device to align in a magnetic field of at least 0.1 Tesla, and more preferably in a magnetic field of at least 0.06 Tesla. One example of a suitable permanent magnetic material includes neodymium-iron-boron (Nd—Fe—B). Suitable permeable magnetic materials include magnetic stainless steel, such as a 303 or 304 stainless steel, Samarium Cobalt, Hiperco or other magnetically responsive elements or blends. A computer controlled magnetic navigation system may be used to apply the magnetic field to a local region of the patient in which the distal tip of the apparatus is in to orient the distal end in a desired direction for advancing the apparatus to the target location. The one or more predetermined distances 32, 34 and 36 of flexible tubing between the magnetically responsive elements 28 allow the distal end of the apparatus 20 to be deflected relative to the longitudinal axis of the tube 22 of the apparatus 20. This allows the apparatus 20 to be guided within the subject's body towards the ostium of a vessel, for example.

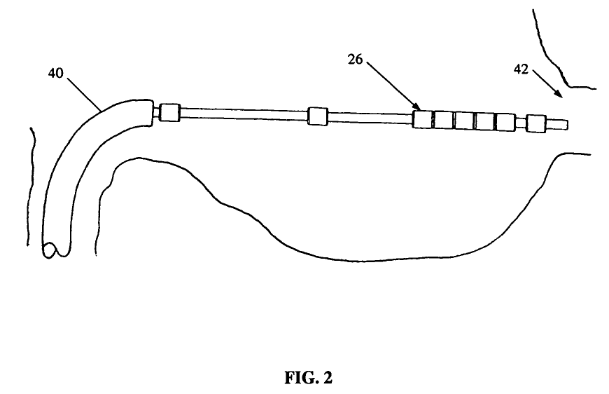

In operation, the apparatus 20 of the present invention may be introduced through a guiding catheter 40 or other thru-lumen catheters and tubing into the subject body's vasculature, and the magnetically responsive elements 28 of the apparatus 20 are aligned by an external magnetic field to orientate the distal tip 26 in a selected direction as shown in FIG. 2. The tip of the apparatus is preferably capable of being deflected a minimum of 10 degrees relative to the longitudinal axis of the apparatus 20, when subjected to a magnetic field having a direction substantially perpendicular to the longitudinal axis of the apparatus 20, wherein the magnetic field is of a magnitude of not more than about 0.1 tesla. Ideally, the tip of the micro-catheter will be preferably deflected 90 degrees when subjected to a magnetic field having a direction perpendicular to the axis of the apparatus. Once the tip has been oriented in the selected direction, the proximal end of the apparatus 24 may then be pushed by hand to advance the distal end 26 though the subject body's vasculature system. Alternatively, the device can be advanced by a mechanical advancer under manual or computer control. The external magnetic field may be changed in orientation to realign or redirect the tip in a stepwise process to continue to steer or guide the catheter though the vasculature system until the distal end is at a selected target location such as the ostium of a vessel 42 as shown in FIG. 2. The apparatus 20 remains in place, and the guide catheter 40 may then be advanced and positioned by the apparatus 20 in the ostium 42 of the target vessel. Guide wires and other medical devices are then able to travel through the inside of the tube 22 out the end to the desired vessel or target area. In the preferred embodiment, the inside diameter of the tube 22 is preferably a minimum of 0.050 millimeters, but may alternately be any diameter sufficient for allowing passage of guide wires or other medical devices.

The magnetically responsive elements 28 also assist in holding the catheter in place to resist back out of the catheter. The magnetically responsive elements 28 will continue to hold the apparatus in alignment with the applied magnetic field direction, which will help in holding the guide catheter in place while the physician advances guidewires or medical devices beyond the guiding catheter 40, for example, to the target location in the subject body. Accordingly, the apparatus 20 therefore facilitates access of the guiding catheter 40 to the target vessel, and also provides support for holding the guiding catheter 40 in place to resist back out of the guiding catheter during a procedure.

The advantages of the above described embodiment and improvements should be readily apparent to one skilled in the art, as to enabling placement and support of a guiding catheter, or other thru-lumen catheters and tubing Additional design considerations such as various methods for securing the magnetically responsive elements to the flexible tube may be incorporated without departing from the spirit and scope of the invention. Accordingly, it is not intended that the invention be limited by the particular embodiment or form described above, but by the appended claims.

What is claimed is:

1. An apparatus for positioning a guide catheter or other thru-lumen catheters and tubing in a target location of the body, the apparatus comprising:
a flexible tube having a proximal end and a distal end, and a lumen therebetween; and
a plurality of magnetically responsive elements disposed on the distal end of the flexible tube, wherein one or more of the magnetically responsive elements are spaced apart from the remaining adjacently positioned magnetically responsive elements located a predetermined distance from the distal end of the flexible tube.

2. The apparatus of claim 1, further comprising an additional magnetically responsive element positioned between the adjacently located magnetically responsive elements and the distal end of the flexible tube.

3. The apparatus of claim 2, wherein the additional magnetically responsive element is located within 20 millimeters of the distal end of the flexible tube.

4. The apparatus of claim 3, wherein the predetermined distance from the distal end of the tube to the adjacently located magnetically responsive elements is in the range of 1 to 20 millimeters.

5. The apparatus of claim 4, wherein one of the one or more spaced apart magnetically responsive elements is proximally spaced from the adjacently located magnetically responsive elements a predetermined distance in the range of about 1 to 20 millimeters.

6. The apparatus of claim 5, wherein one of the one or more spaced apart magnetically responsive elements is proximally spaced from the adjacently located magnetically responsive elements a predetermined distance in the range of about 10 to 40 millimeters.

7. The apparatus of claim 6, wherein the remaining adjacently located magnetically responsive elements are made of such material and are of such dimensions that under the influence of an applied magnetic field, the distal end portion of the apparatus substantially aligns with the local applied magnetic field direction.

8. The apparatus of claim 7, wherein the predetermined distances provide regions of flexibility for enabling the distal end of the apparatus to deflect a minimum angle from the longitudinal axis of the tube when subjected to an external magnetic field.

9. The apparatus of claim 8, wherein the tip of the apparatus is capable of being deflected a minimum of 10 degrees relative to the longitudinal axis of the tube, when subjected to a magnetic field having a direction perpendicular to the longitudinal axis of the tube and a magnitude of not more than about 0.1 Tesla.

10. An apparatus for positioning a guide catheter or other thru-lumen catheters and tubing in a target area of the body, the apparatus comprising:
a flexible tube having a proximal end and a distal end and a lumen therebetween; and
a plurality of magnetically responsive elements disposed around the distal end of the flexible tube, wherein a predetermined number of the magnetically responsive elements are located adjacent each other at a minimum distance from the distal end of the tube, and one or more magnetically responsive elements are proximally spaced from the predetermined number of magnetically responsive elements by one or more predetermined distances.

11. The apparatus of claim 10, wherein the plurality of magnetically responsive elements are made of such material and are of such dimensions that under the influence of an applied magnetic field, the distal end portion of the apparatus substantially aligns with the local applied magnetic field direction.

12. The apparatus of claim 10, further comprising an additional magnetically responsive element positioned between the predetermined number of adjacently located magnetically responsive elements and the distal end of the flexible tube.

13. The apparatus of claim 12, wherein the additional magnetically responsive element is located within 1 millimeter of the distal end of the flexible tube.

14. The apparatus of claim 13, wherein the minimum distance from the distal end of the tube to the predetermined number of magnetically responsive elements is about 10 millimeters.

15. The apparatus of claim 14, wherein one of the one or more spaced apart magnetically responsive elements is proximally spaced from the predetermined number of magnetically responsive elements by a predetermined distance in the range of about 1 to 30 millimeters.

16. The apparatus of claim 15, wherein one of the one or more spaced apart magnetically responsive elements is proximally spaced from the predetermined number of magnetically responsive elements by a predetermined distance in the range of about 10 to 40 millimeters.

17. The apparatus of claim 16, wherein the predetermined number of magnetically responsive elements are made of such material and are of such dimensions that under the influence of an applied magnetic field, the distal end portion of the apparatus substantially aligns with the local applied magnetic field direction.

18. The apparatus of claim 11, wherein the predetermined distances provide regions of flexibility for enabling the distal end of the apparatus to deflect a minimum angle from the longitudinal axis of the tube when subjected to an externally applied magnetic field.

19. The apparatus of claim 18, wherein the tip of the apparatus is capable of being deflected a minimum of 10 degrees relative to the longitudinal axis of the tube, when subjected to a magnetic field having a direction perpendicular to the longitudinal axis of the tube and a magnitude of not more than about 0.1 Tesla.

20. The apparatus of claim 19, wherein the size and length of each of the magnetically responsive elements is preferably not more than 20 millimeter in diameter and in the range of 0.05 to 20 millimeter in length.

21. The apparatus of claim 20, wherein the magnetically responsive elements are secured to the tube by an adhesive or other suitable bonding method such as heat shrink, fusion, for example.

22. An apparatus for positioning a guide catheter or other thru-lumen catheters and tubing in a target area of the body, the apparatus comprising:
a flexible tube having a proximal end and a distal end and a lumen therebetween; a predetermined number of the magnetically responsive elements disposed around the tube and located adjacent to each other at a minimum distance from the distal end of the tube; and
one or more magnetically responsive elements proximally spaced from the predetermined number of magnetically responsive elements by one or more predetermined distances, wherein the predetermined distances provide regions of flexibility for enabling the distal end of the apparatus to be deflected a minimum angle when subjected to an externally applied magnetic field.

23. The apparatus of claim 22, wherein each of the magnetically responsive elements are made of such material and are of such dimensions that under the influence of an applied magnetic field, the distal end portion of the apparatus substantially aligns with the local applied magnetic field direction.

24. The apparatus of claim 23, wherein the tip of the apparatus is capable of being deflected a minimum of 10 degrees relative to the longitudinal axis of the tube, when subjected to a magnetic field having a direction perpendicular to the longitudinal axis of the tube and a magnitude of not more than about 0.1 Tesla.

25. The apparatus of claim 24, wherein the size and length of each of the magnetically responsive elements is preferably not more than 20 millimeter in diameter and in the range of 1 to 20 millimeter in length.

26. The apparatus of claim 25, wherein the magnetically responsive elements are secured to the tube by an adhesive or other suitable bonding methods.

27. The apparatus of claim 22, further comprising an additional magnetically responsive element positioned between the predetermined number of adjacently located magnetically responsive elements and the distal end of the flexible tube.

28. The apparatus of claim 27, wherein the additional magnetically responsive element is located within 1 millimeters of the distal end of the flexible tube.

29. The apparatus of claim 22, wherein the minimum distance from the distal end of the tube to the predetermined number of magnetically responsive elements is about 1 millimeter.

30. The apparatus of claim 29, wherein one of the one or more spaced apart magnetically responsive elements is proximally spaced from the predetermined number of magnetically responsive elements by a predetermined distance in the range of about 1 to 30 millimeters.

31. The apparatus of claim 30, wherein one of the one or more spaced apart magnetically responsive elements is proximally spaced from the predetermined number of magnetically responsive elements by a predetermined distance in the range of about 1 to 30 millimeters.

32. The apparatus of claim 31, wherein the predetermined number of magnetically responsive elements are made of such material and are of such dimensions that under the influence of an applied magnetic field, the distal end portion of the apparatus substantially aligns with the local applied magnetic field direction.

33. The apparatus of claim 23, wherein the magnetically responsive elements are preferably made of neodymium-iron boron or other suitable elements or blends.

34. An apparatus for positioning a guide catheter or other thru-lumen catheters and tubing in a target location of the body, the apparatus comprising:
a flexible element having a proximal end and a distal end,
at least one magnetically responsive element disposed on the flexible element spaced from, but adjacent to, the distal end;
a plurality of magnetically responsive elements disposed on the flexible element, proximal to and spaced a predetermined distance from the at least one magnetically responsive element, each of the plurality of magnetically responsive elements being located closely adjacent to each other.

35. The apparatus according to claim 34 wherein the flexible element is tube, having a lumen therethrough.

36. The apparatus according to claim 34 further comprising at least one auxiliary magnetically responsive element on the flexible element, proximal to the plurality of magnetically responsive elements.

37. The apparatus according to claim 36 wherein there are at least two auxiliary magnetically responsive elements on the flexible element, each spaced proximally from the plurality of magnetically responsive elements, and wherein one of the at least two auxiliary magnetically responsive elements is spaced from the other of the at least two auxiliary magnetically responsive elements.

38. In combination with a medical catheter having a lumen therein, a guide apparatus adapted to fit in the lumen of the medical catheter, the guide apparatus comprising:
a flexible element having a proximal end and a distal end,
at least one magnetically responsive element disposed on the flexible element spaced from, but adjacent to, the distal end;
a plurality of magnetically responsive elements disposed on the flexible element, proximal to and spaced a predetermined distance from the at least one magnetically responsive element, each of the plurality of magnetically responsive elements being located closely adjacent to each other.

39. The combination according to claim 38 wherein the flexible element is tube, having a lumen therethrough.

40. A method of navigating a medical catheter having a lumen therein into a vascular branch in a subject's body, the method comprising applying a magnetic field to a guide apparatus comprising a flexible element having a proximal end and a distal end, at least one magnetically responsive elements disposed on the flexible element spaced from, but adjacent to, the distal end; and a plurality of magnetically responsive elements disposed on the flexible element, proximal to and spaced a predetermined distance from the at least one magnetically responsive element, each of the plurality of magnetically responsive elements being located closely adjacent to each other, and advancing the guide apparatus into the vascular branch, and
advancing the medical catheter over the guide apparatus and into the branch, which maintaining the externally applied magnetic field.
